(19) United States
(12) Patent Application Publication
(10) Pub. No.: US 2017/0175066 A1
(43) Pub. Date: Jun. 22, 2017

(54) SINGLE USE BAG FOR BIOLOGICAL MATERIALS AND PROCESSING

(71) Applicant: E I DU PONT DE NEMOURS AND COMPANY, Wilmington, DE (US)

(72) Inventors: REBECCA L. OLSEN, WILMINGTON, DE (US); DIANE MCCAULEY HAHM, BOOTHWYN, PA (US)

(21) Appl. No.: 15/368,941

(22) Filed: Dec. 5, 2016

Related U.S. Application Data
(60) Provisional application No. 62/269,123, filed on Dec. 18, 2015.

(51) Int. Cl. C12M 1/00; A61F 5/44; A61J 1/10

(52) U.S. Cl. CPC C12M 23/14; C12M 23/28; C12M 23/26; A61J 1/10; A61F 5/44

(57) ABSTRACT

A single-use bag includes a collapsible a body having a first end and an opposing second end bounding a compartment. The collapsible body comprises at least one flexible sheet having an interior surface and an exterior surface; wherein the interior surface layer comprises low density polyethylene having a melt index from 0.9 to 2.0 g/10 min with a weight average molecular weight (Mw) greater than 200,000 Da, and an Mz greater than 1,500,000 Da. Optionally, the flexible sheet comprises an oriented film as an additional layer on the exterior surface. The single-use bag is useful in biological processing, including use in a disposable cell culture bioreactor.

### CROSS-REFERENCE TO RELATED APPLICATION

The present application claims priority under 35 U.S.C. §119 to U.S. Provisional Application No. 62/269,123, filed on Dec. 18, 2015, which is incorporated herein by reference in its entirety.

### FIELD OF THE INVENTION

Provided herein are a method and a system for containing and processing biological materials. More particularly, disposable single-use components and systems are useful for containing and processing biological materials.

### BACKGROUND OF THE INVENTION

Several patents and publications are cited in this description in order to more fully describe the state of the art to which this invention pertains. The entire disclosure of each of these patents and publications is incorporated by reference herein.

A variety of vessels are available for storing or manipulating biological materials and fluids; for carrying out chemical, biochemical or biological reactions; and for furthering sterile and non-sterile mixing applications. Modern cell cultivation is typically accomplished using a bioreactor or a fermenter vessel. Despite the fact that a bioreactor and a fermenter are essentially similar in design and general function, the dichotomy in nomenclature is sometimes used to distinguish between animal and plant cell culture. As used herein, the term “bioreactor” refers to an apparatus for aerobic or anaerobic cultivation of cells that are microbial, animal, insect or plant cells. Thus, the term “bioreactor” encompasses a fermenter.

Traditionally, the biopharmaceutical industry has produced and manufactured products in stainless steel or glass reactors and containers. After each use (typically 3 to 15 days), the reactor and its components may require one or more of disassembly, cleaning, reassembly, reconfiguration or autoclaving before reuse. This can be a time consuming, laborious process requiring the manipulation of many heavy components or many small and fragile components. Additionally, the cleaning procedure must generally be validated, to ensure that it is completed correctly each time with the same consistent results. At best, after all the work has been completed, the reactor and its components have been rendered aseptically clean. Nevertheless, contamination may occur via residual organisms or adventitious organisms that enter the aseptic assembly.

Many designs have attempted to overcome these issues by using disposable liners in the glass or stainless tank. For example, U.S. Pat. No. 6,245,555 describes a plastic liner that is inserted into an existing tank to reduce the amount of cleaning and increase the level of sterility.

Such liners have limitations, however. For example, the liner must conform to the inner surface of the tank to prevent any discontinuity in the circulation within the device or to prevent the formation of dead spots or pockets in which material may get trapped and fester or create uneven flow throughout the system. Wrinkles in the liner may create similar problems.

Rigid or semi-rigid molded plastic containers have also been proposed as drop-in replacements for stainless steel bioreactors (e.g., U.S. Pat. No. 8,999,702). Although described as disposable single-use reactors, such containers require complex molding processes for their production and take up significant storage space in the production facility and in shipping. After use, these reactors also take up significant space in landfills unless special arrangements are made for recycling them.

Increasingly, disposable single-use bags (hereinafter referred to as “SUBs” or “SUB”) and other process containers comprising flexible plastic sterile bags are used without insertion into rigid tanks. The SUBs are made by converting multilayer barrier films into sealed bags of different sizes (e.g., 500 ml to 2,000 liters). Bags can be used for bioreactors, cell culture, storage of media, sterile water for injection (WFI), or buffer solutions, and for storage, final filling, mixing or final drug packaging of biological products.

Disposable SUB technology can be obtained from the manufacturer in multiple, flexible configurations. In addition, SUB offer advantages over designs utilizing glass and stainless steel, such as reduced turn-around time, lower capital investment, reduced need for validation of sterility, lower risk of cross contamination, and reduced need for supporting infrastructure.

Representative SUB bags, equipment and accessories are described in U.S. Pat. Nos. 8,556,111; 8,556,497; 8,840,299; 8,894,756; and 9,109,193; and in U.S. Patent Application Publications US2005/0282269; US2008/0130405; US2008/0131960; US2010/0203624; US2011/0151551; US2011/0151552; US2011/0201100; US2014/0011270; and US2014/0349385.

The most critical polymeric material in a disposable SUB is in direct contact with its contents. For example, the contact layer may include extractable materials that can migrate from the polymer into the contents. It has recently been discovered that derivatives of a specific antioxidant present in incumbent contact resins, mainly PE-type resins, was significantly impacting cell growth and cell viability (see, e.g., Hammond M., et al., “Identification of a Leachable Compound Detrimental to Cell Growth in Single-Use Bioprocess Containers,” PDA J Pharm Sci Technol, Vol. 67, No. 2, 2013, pp. 123-134.) Thus, the extractable profile of contact resins is an important current topic in the industry.

In addition, disposable SUBs are usually sterilized with radiation, for example gamma rays or electron beam radiation, or with aggressive chemicals such as ethylene oxide (ETO). They may also be sterilized by steam. Therefore, the materials from which the SUB is fabricated preferably do not degrade upon exposure to these sterilization methods.

Therefore, it is desirable to provide a disposable SUB with a minimal total amount of extractable material, such as antioxidants or other polymer additives, that can migrate into the contents of the SUB. Also preferably, the disposable SUB does not degrade substantially when exposed to common sterilization procedures.

### SUMMARY OF THE INVENTION

Provided herein is a system for containing or processing biological materials, including single-use biopharma processes, and methods of using the system.

Further provided is a single-use flexible film bag for containing or processing biological materials comprising: a body having a first end and an opposing second end bounding a compartment, the body comprising at least one flexible sheet; the bag having an interior surface and an exterior surface; wherein the interior surface comprises low density polyethylene having a melt index from 0.8 to 2.5 g/10 min with a weight average molecular weight (Mw) greater than 200,000 Da, and an Mz greater than 1,500,000 Da.

In one embodiment of the single-use flexible film bag, the flexible sheet comprises a multilayer film adhered to a substrate comprising a monoaxially or biaxially oriented film, and the film is optionally coated with a barrier-enhancing agent.

Further provided is a method for preparing a biological product, the method comprising:

providing a single-use bioreactor comprising the single-use flexible film bag described above;

placing a culture medium and a biological sample inside the single-use flexible film bag; and

allowing the biological sample to interact with the culture medium to provide a cell culture medium.

The method may further comprise removing at least a portion of the cell culture medium from the single-use flexible film bag; and transforming the portion of the cell culture medium to obtain the biological product.

Further provided is a biological product obtained by placing a culture medium and a biological sample inside the compartment of a single-use flexible film bag as described above, and allowing the biological sample to interact with the culture medium to provide a cell culture medium containing the biological product, optionally further comprising wherein at least a portion of the cell culture medium is transformed to obtain the biological product.

### DETAILED DESCRIPTION OF THE INVENTION

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present). As used herein, the terms “a” and “an” include the concepts of “at least one” and “one or more than one”. The word(s) following the verbs “is” or “are” can be a definition of the subject.

The term “consisting essentially of” when used in reference to polymer compositions indicates that substantially (greater than 95 weight % or greater than 99 weight %) the only polymer(s) present in a composition are the polymer(s) recited. Thus, this term does not exclude the presence of impurities or additives, e.g. conventional additives. Moreover, such additives may possibly be added via a master batch that may include other polymers as carriers, so that minor amounts (less than 5 or less than 1 weight %) of polymers other than those recited may be present. Any such minor amounts of these materials do not change the basic and novel characteristics of the composition.

When the term “about” is used in describing a value or an end-point of a range, the disclosure should be understood to include the specific value or end-point referred to.

Unless stated otherwise, all percentages, parts, ratios, etc., are by weight. Further, when an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range. When a component is indicated as present in a range starting from 0, such component is an optional component (i.e., it may or may not be present). When present an optional component may be at least 0.1 weight % of the composition or copolymer, unless specified at lower amounts.

When materials, methods, or machinery are described herein with the term “known to those of skill in the art”, “conventional” or a synonymous word or phrase, the term signifies that materials, methods, and machinery that are conventional at the time of filing the present application are encompassed by this description. Also encompassed are materials, methods, and machinery that are not presently conventional, but that may have become recognized in the art as suitable for a similar purpose.

As used herein, the term “copolymer” refers to polymers comprising copolymerized units resulting from copolymerization of two or more comonomers and may be described with reference to its constituent comonomers or to the amounts of its constituent comonomers such as, for example “a copolymer comprising ethylene and 15 weight % of methyl acrylate”. A description of a copolymer with reference to its constituent comonomers or to the amounts of its constituent comonomers means that the copolymer contains copolymerized units (in the specified amounts when specified) of the specified comonomers. Polymers having more than two types of monomers, such as terpolymers, are also included within the term “copolymer” as used herein. A dipolymer consists essentially of two copolymerized comonomers and a terpolymer consists essentially of three copolymerized comonomers. The term “consisting essentially of” in reference to copolymerized comonomers allows for the presence of minor amounts (i.e. no more than 0.5 weight %) of non-recited copolymerized units, for example arising from impurities present in the commoner feedstock or from decomposition of comonomers during polymerization.

As used herein the term “biopharma process” refers to a process for producing a biological product that is useful as a biopharmaceutical. The terms “biological sample” and “biological material” are synonymous and used interchangeably herein to refer, without limitation, to any particle, substance, extract, mixture, or assembly derived from or corresponding to one or more organisms, cells, or viruses. The term “biological product” refers to a compound, substance or material that is obtained from a biological process such as fermentation, cell cultivation, enzymatic processes or the like. The term “biopharmaceutical” refers to a biological product that is useful for treating an illness or disorder in humans or other organisms.

Cells that may be cultured in an automated cell management system include, without limitation, one or more of animal cells, insect cells, mammalian cells, human cells, transgenic cells, bacterial cells, yeast cells, genetically engineered cells, transformed cells, cell lines, plant cells, anchorage-dependent cells, anchorage-independent cells, and other cells capable of being cultured in vitro.

The term “culture medium” refers to additional components that facilitate analysis or provide nutrients to the biological sample, such as fluid (e.g., water), buffer, culture nutrients, salt, other reagents, dyes, and the like. Accordingly, the biological sample may include one or more cells disposed in a culture medium. The term “cell culture medium” refers to the combination of the culture medium and the biological sample. The term “transform” refers to operations that facilitate isolation or purification of a biological product from a cell culture medium. Such operations include for example, denaturation, precipitation, filtration, evaporation, sublimation, freeze-drying, distillation, crystallization, chromatography, electrophoresis and the like. It may also refer to chemical reactions such as neutralization, saponification, acidification, acylation and the like.

Alternatively, the term “culture medium” refers to a liquid solution that provides nutrients (e.g., vitamins, amino acids, essential nutrients, salts, and the like) and properties (e.g., similarity, buffering) to maintain living cells (or living cells in a tissue) and support their growth. Also alternatively, the term “cell culture medium” as used herein refers to tissue culture medium that has been incubated with cultured cells in forming a cell culture; and more preferably refers to tissue culture medium that further comprises substances secreted, excreted or released by cultured cells, or other compositional or physical changes that occur in the medium resulting from culturing the cells in the presence of the tissue culture medium. Some suitable tissue culture media are commercially available.

The terms “bioreactor” and “bioreactor vessel” are synonymous and used interchangeably herein to refer to any apparatus, such as a fermentation chamber or fermenter, for growing organisms such as bacteria or yeast under controlled conditions for production of substances such as pharmaceuticals, antibodies, or vaccines, or for the bioconversion of organic waste.

As used herein, the term “cell culture” means a group of cells that are simultaneously undergoing one or more processes such as multiplication, growth, maintenance, differentiation, transfection, or propagation of cells, tissues, or their products.

As used herein, the term “sensor” or “probe” means, but is not limited to, mechanical, electrical or optical sensing or probing devices that measure information such as physiologically relevant information (e.g., mixing rate, gas flow rate temperature, humidity, pressure, pH, biochemicals such as glucose, glutamine, lactic acid, ammonia, and nitrogen, biomolecules, dissolved gases such as oxygen, CO₂, and other chemical parameters, enzyme-based parameters, radiation, magnetic and other physical parameters), or other information or parameters. The sensors/probes may be optical probes which present the output in a visual manner.

The term “(meth)acrylic acid” refers to acrylic acid, methacrylic acid, or combinations thereof. Likewise, the term alkyl (meth)acrylate refers to alkyl acrylate, alkyl methacrylate or combinations thereof, wherein the alkyl group comprises one to eight, preferably one to four, carbon atoms.

A flexible film single-use bag (SUB) is used in a biopharma process. It is desirable that all single-use containers that contain any inputs or outputs of biopharma processes maintain the same low extractable profile. SUBs include bags for use in bioreactors, cell culture, storage of media, sterile water for injection (WFI) or buffer solutions, or storage, final filling, mixing and final drug packaging of biological products.

The SUB comprises a flexible film as described in more detail below formed into a bag or pouch shape. Flexible film bags can be prepared by combinations of folding and overlaying one or more portions of flexible film and joining the flexible film at the overlaid portions by, for example, heat sealing, radio frequency (RF) welding, ultrasonics, adhesive bonding, etc. Simple bags may be prepared using automated bag making machines.

Although the following description refers specifically to preparation of a flexible film bag useful in a bioreactor comprising a mixing bag assembly, other bags of the invention can be prepared using similar techniques. Depending on their intended use in a biopharma process, the bags may have various combinations of ports, fitments, attachments and the like as described below.

The mixing bag assembly comprises a flexible and collapsible bag-like body having an interior surface and an exterior surface. The interior surface bounds a compartment. More specifically, the body comprises a side wall that, when the body is unfolded, has a substantially circular or polygonal transverse cross section that extends between a first end and an opposing second end. The first end terminates at a top end wall while the second end terminates at a bottom end wall.

In one embodiment, the body of the mixing bag assembly comprises a two-dimensional pillow style bag wherein two sheets of material are placed in overlapping relation and the two sheets are bounded together at their peripheries to form the internal compartment. Alternatively, a single sheet of material can be folded over and seamed around the periphery to form an internal compartment. In other embodiments, the body comprises a three-dimensional bag which not only has an annular side wall but also a two-dimensional top end wall and a two-dimensional bottom end wall.

The three dimensional body may comprise a plurality, i.e., typically three or more, discrete panels attached together to form for a mixing bag. For example, the body may comprise four panels, i.e., a top panel, front panel, back panel, and bottom panel. Each of the panels has a substantially square or rectangular central portion. The top panel and the bottom panel include a first end portion and an opposing second end portion projecting from opposing ends of the central portion. Each of the end portions has a substantially trapezoidal configuration with opposing tapered edges. The front panel and back panel each include a triangular first end portion and an opposing triangular second end portion projecting from opposing ends of the central portion. Corresponding perimeter edges of each panel are seamed together so as to form a substantially box shaped body. The panels are seamed together using methods known in the art such as heat sealing, RF welding, ultrasonics, other sealing energies, adhesives, or other conventional processes. It is appreciated that by altering the size and configuration of some or all of the panels, the body of the mixing bag can be formed having a variety of different sizes and configurations. It is also appreciated that any number of panels can be used to adjust the size and configuration of the mixing bag body.

In still other embodiments, the body of the mixing bag can be formed by initially extruding or otherwise forming a polymeric sheet in the form of a continuous tube. In one embodiment, the tube can simply be cut to length and each end seamed closed to form a two-dimensional pillow style bag. In an alternative embodiment, each end can be folded like the end of paper bag and then seamed closed so as to form a three dimension body. In still another embodiment, a length of tube can be laid flat so as to form two opposing folded edges. The two folded edges are then inverted inward so as to form a pleat on each side. The opposing ends of the tube are then seamed closed. Finally, an angled seam is formed across each corner so as to form a three dimensional bag when filled.

It is appreciated that the above techniques can be mixed and matched with one or more polymeric sheets and that there are still a variety of other ways in which the body can be formed having a two or three dimensional configuration. Further disclosure with regard to one method of manufacturing three-dimensional bags is disclosed in U.S. patent application publication US2002/0131654.

The body of the mixing bag can be manufactured to have virtually any desired size, shape, and configuration. For example, body can be formed having compartment sized to hold up to 0.5 liter, 1 liter, 5 liters, 25 liters, 50 liters, 100 liters, 200 liters, or other desired amounts.

In any embodiment, it is desirable that when the mixing bag body is filled it is sufficiently supported, either as a free-standing self-supporting assembly, or by a structure that surrounds or supports at least a portion of the mixing bag body. Having at least generally uniform support of the mixing bag body helps to preclude failure of the body by hydraulic forces applied to the mixing bag body when filled with a solution. In some embodiments, the mixing bag may be constructed of sufficiently strong flexible films so that it is self-supporting when filled with solution so that it can be placed on a table or bench to support the floor of the bag without additional supporting framework for the side walls of the bag. In such embodiments, the side walls of the bag body when filled may slump due to gravity to provide a pillow-like shape to the filled mixing bag. The side walls of the mixing bag may be constructed with one or more panels configured to facilitate the slump while minimizing hydraulic pressure on the overall bag structure.

In other embodiments, however, the mixing bag body can be specifically configured to be complementary or substantially complementary to a supporting framework or container. The body is constructed of flexible sheets allowing it to conform to the configuration of any supporting framework as it is filled with solution. For example, the mixing bag may have a substantially box-shaped configuration and may be placed in a box-like cabinet that provides support to the floor and side walls of the bag and the mixing bag body. In still other embodiments, the bag may be placed in a container or tank that supports the floor and side walls of the bag.

The mixing bag may have a more complex shape, such as those described in U.S. Patent Application Publications US2008/0131959 and US2008/0131960.

The mixing bag assembly may further comprise at least one port mounted on the body so as to communicate with the compartment of the body. Ports are a necessary feature of SUBs for delivering controlled volumes of gas, liquid, or other material to growth media containing cells; for sampling sample fluid out of the bioreactor; for extracting material out of the bioreactor; and for inserting probes, such as a temperature probe, to monitor conditions within the SUB. The design of each port depends on the desired function of the port. It is appreciated that any number of ports can be formed on the body of the bioreactor and that a variety of different types and sizes of ports can be used depending on the type of material to be dispensed into the interior compartment and how the material is to be dispensed therefrom.

Ports may comprise fitments or connectors fabricated from thermoplastic materials or metals and inserted into holes in the bioreactor bag material. One or more ports can be located in the bag and sealingly isolated from the environment by sterile means. For example a connector may be fabricated to accept tubing or a container mechanically attached thereto, such as by including a stem that may be barbed or screw threaded. A plastic hose may extend from such hose barb that can be sealed such as by clamps, interlocking fittings such as Luer fittings, or a weldment at the end remote from the bag at which the port is attached. In some embodiments, the port may be recloseable, such as by incorporating a septum suitable for piercing by a hollow needle, a snap cap, a screw cap, a valve or other methods. The ports may also comprise screens, filters, selectively permeable membranes or the like to allow some materials to pass through the ports while preventing other materials from passing through. They are desirably made from materials suitable for attaching to the bioreactor bag material by, for example, heat sealing, RF welding, ultrasonic welding, adhesive bonding, etc., and also compatible with the method for sterilizing the bioreactor bag. For example, ports comprising ethylene vinyl acetate or polyethylene (LLDPE or LDPE) are readily sealable to the low density polyethylene of the sealant layer described below. Notable ports, fitments, connectors or tubing comprise a surface layer comprising LDPE that does not contain an antioxidant, such as DuPont™ 20 Series DPE-20. Additionally or alternatively, they may be attached to the bioreactor bag by mechanical means, such as a two-piece fitment held in place by friction fitting, snap fitting, screw threading, and the like.

For example, a port may comprise a barbed tubular stem having a flange outwardly projecting from an end thereof. During assembly of the bag, a plurality of holes corresponding to the desired number of ports may be made through the top or side panel(s) prior to complete seaming together of the panels. The stem of each port is then passed through a corresponding hole until the flange rests against the panel. Conventional welding or other sealing techniques are then used to seal each flange to the panel. During use, the barbed stem is selectively coupled with a tube or container for delivering material into or out of the interior compartment or the bioreactor.

Alternatively or additionally, an outlet port or drain may be located at the bottom of the bioreactor bag assembly to allow for removal of at least a portion of the contents of the bioreactor for assay, isolation or purification. The outlet port preferably has a closure function so that the contents can be removed when needed. The outlet port may comprise a filter or membrane, so that liquid may be removed selectively while solid materials are retained inside the bag. The outlet port may be incorporated into the bottom of the bag in a manner similar to that described above.

The mixing bag may incorporate a gas distribution device such as those described in U.S. Patent Application Publication US2005/0282269 and U.S. Pat. Nos. 5,565,015 and 6,432,698.

The mixing bag may incorporate a vortex breaker in the form of one or more plastic sheet materials that are attached to various inner surfaces of the bag and disrupt the formation of vortices within the bag. Desirably, the sheet(s) are formed of the same material as the bag and are sealed to the bag surfaces. Alternatively, the sheets may be formed of a monolayer film comprising the same material as, or a material which can be bonded to, that used in the contact layer of the film used for the mixing bag. Each sheet of the vortex breaker has a first end and a second end, the first end being attached to a first inner surface of the bag and the second end being attached to a second inner surface of the bag which is at different location in the bag from the first inner surface with the one more sheets of the vortex breaker extending between the first and second inner surfaces of the bag. Preferably, the sheet(s) extend across a cross-dimension (such as the diameter or the width) of the bag. The sheets may be adhered to the inside of the mixing bag by, for example, heat sealing, RF welding, ultrasonics, adhesive bonding, etc., preferably prior to final assembly of the mixing bag. Preferably, the sheet(s) are perforated with one or more slits or openings to allow for good flow and mixing without a vortex being formed. Embodiments of such vortex breakers are described in U.S. Pat. No. 8,556,497.

In any type of bioreactor, the conditions must be closely monitored by sensors and controlled to provide ongoing optimum conditions within the bioreactor, such that microorganisms, cells, and the like perform their desired functions successfully. Conditions that may be monitored include, without limitation, one or more chemical, biochemical, nutritional, biological and environmental conditions, for example one or more of gas (i.e., air, oxygen, nitrogen, carbon dioxide, ammonia), glucose, specific protein levels, and any number of other parameters either required or produced by cellular metabolism inside the SUB flow rates, temperature, pH and dissolved oxygen levels, and agitation speed/circulation rate. In some embodiments, sensors and probes to monitor the conditions may be inserted into the mixing bag through one or more of the ports described above during use.

Each connection into the bioreactor increases the likelihood of contamination. Typical SUB bioreactor systems may allow a maximum of four insertion points into the bioreactor. However, in GMP manufacturing environments it is often required to have a redundant sensor in case of failure or drift.

To address such difficulties, the bioreactor mixing bag may comprise a disposable manifold system for use in coupling sensors, fluid samplers, conduits, and the like, to the cell culture bioreactor in a sterile manner. The disposable bioreactor manifold system may include an externally attachable bioreactor manifold connector body for fluidly attaching modular sensor arrangements that measure physical variables and other parameters of medium contained within a bioreactor, as well as medium sampling components, and other connections, as well as at least one conduit fluidly connector connecting the bioreactor manifold connector body with a pump for pumping fluids between the bioreactor and the bioreactor manifold connector body.

The disposable single-use cell culture bioreactor manifold system may be assembled by configuring any number of modular components, such as connections, sensors, samplers, additional lines, and conduits, fluidly connected or coupled to a bioreactor manifold connector body which can be externally attached and fluidly connected to a port on a bioreactor, either directly or indirectly by way of a conduit or the like, in order to interact with a liquid medium flowing through the manifold connector body. Each manifold system may be configured for the optimum number of components for a particular bioreaction. In this way, the design of the bioreactor mixing bag may be made more generally applicable and its versatility increased, by not having to be configured for a specific bioreaction. The system advantageously simplifies design, lessens the possibility of contamination of the bioreactor, and lowers the cost of monitoring, testing and supporting of bioreactor vessels. Embodiments of such bioreactor manifold systems are described in U.S. Patent Application Publication US2011/0201100.

During use of a SUB, a constant movement of the cells in the reactor helps to provide a constant mixing of the contents. Accordingly, it is desirable for the bioreactor to accommodate means for mixing the contents. One system for a SUB has been to use a large table equipped with motors or hydraulics onto which a bioreactor bag is placed. The motors/hydraulics rock the bag, providing constant movement of the cells. See U.S. Pat. No. 6,191,913. Advantageously, this system does not require special provisions to the mixing bag itself. However, such a system requires the use of capital-intensive equipment, with components that are susceptible to wear. Additionally, the size of the bag that can be used with the table is limited by the size of table and the lifting capability of its motors/hydraulics.

Other alternatives have been developed in which the mixing bag is modified to accommodate means for mixing. For example, an alternative system uses a long flexible tube-like bag that has both ends attached to movable arms such that the bag after filling is suspended downwardly from the movable arms in the shape of a “U”. The arms are then alternately moved upward or downward relative to the other so as to cause a rocking motion and fluid movement within the bag. If desired the midsection may contain a restriction to cause a more intimate mixing action. Other similar systems suspend the mixing bag in an “inverted U” or “arch” arrangement in (U.S. Patent Application Publications US2008/0131959 and US2008/0131960).

Another system uses one or more bags that are capable of being selectively pressurized and deflated in conjunction with a disposable bioreactor bag, as disclosed in U.S. Patent Application Publications US2008/0130405. The pressure bag(s) may surround a selected outer portion of the bag or may be contained within an inner portion of such a bag. By selectively pressurizing and deflating the pressure bag(s), fluid motion in the bag ensures cell suspension, mixing or gas or nutrient/excrement transfer within the bag without damaging shear forces or foam generation.

Preferably, two or more pressure bags are used at or near the opposite ends of the bag. Each pressure bag has an inlet and an outlet that can be selectively opened or closed. An air supply is provided to the inlet of the pressure bag. Optionally, a vacuum supply is provided to the outlet. As one pressure bag is inflated by closing the outlet and opening the inlet, the other is deflated by closing the inlet and opening the outlet. This inflation/deflation applies a pressure to one end of the bag compressing the fluid in that end and moving it toward the end at which the pressure is less. By alternating the inflation/deflation in the opposite pressure bags, one obtains a wave or rocking movement of the fluid throughout the bag. Alternative embodiments of this system comprise pressure bag(s) external to the mixing bag or internal to the mixing bag. In either embodiment, the pressure bag(s) may be secured to the mixing bag provided to prevent it from moving off the bag. Straps, hook and loop attachment tapes, adhesives, heat bonding and the like may be used as the attachment means. The mixing bag may need to be fabricated to include such means. For example, the pressure bags may be heat sealed onto at least a portion of the mixing bag. Further when one or more pressure bag is internal to the mixing bag, inlet and outlet ports for each pressure bag need to be provided through the mixing bag.

The mixing bag may alternatively comprise a fitting that provides or accommodates means for mixing the contents of the bag. They are desirably made from materials suitable for attaching to the bioreactor bag material by, for example, heat sealing, RF welding, adhesive bonding, etc., and also compatible with the method for sterilizing the bioreactor bag. The fitting or mixing means preferably are incorporated into the mixing bag during assembly so that they may be sterilized at the same time as the mixing bag.

The mixing bag may have a magnetic mixing element such as a magnetic stir bar or impeller with one or more blades disposed within it preferably at or near the bottom of the bag. Disposed below the bag and adjacent to the mixing element is a magnetic drive and in magnetic communication with the mixing element that rotates the magnetic mixing element. This allows the element with impellers to be isolated and kept sterile within the bag while being driven by the external drive through magnetic coupling of the mixing element and the drive. An advantage of magnetically-driven mixing elements is that the mixing element and the magnetic drive are in magnetic communication and need not be mechanically linked. This allows for the mixing element to be sealed to the inside surface within the mixing bag without needing a hole through the mixing bag film. Alternatively, the mixing element may protrude through a hole into the interior of the mixing bag and is sealed with a flange either to the inside surface or the outside surface of the mixing bag. Embodiments of magnetic mixing fittings are described in U.S. Pat. Nos. 7,278,780 and 8,556,497.

Another embodiment of the bag uses a mixer that is driven by a shaft from an external source such as a motor or magnetic drive. The mixer has one or more impellers or blades mounted to one or more sections of the shaft. The shaft is rotated or oscillated to move the impeller blades through the contents of the mixing bag. The impeller shaft may enter the mixing bag from the top or from the bottom. The impellers may be positioned on the lower portion of the shaft or they could also be located on an upper portion of the shaft as well as or in lieu of the lower impellers so long as one achieves the desired mixing without vortex formation. To accommodate the mixing shaft, the bag includes a fitting that is attached to the end of the bag. The fitting may be attached by being ultrasonically welded, by adhesive or other attachment means. The fitting may be of any suitable material, including plastic or metal. The fitting supports the mixing shaft within the bag during use, and may comprise lower bearings and upper bearings that are known in the art such as by U.S. Pat. No. 7,547,135, which support the impeller shaft for rotation. These bearings may be any suitable type of bearing including metal bearings or plastic bearings, but since they may come in contact with the fluid to be mixed are preferable selected to be dry running bearings. At the end of the shaft external to the mixing bag, a drive system such as an electric motor or magnetically coupled drive mechanism is provided to rotate the shaft. The drive system can be mounted onto the mixing assembly via a bayonet bracket that slides onto the outer surface of the fitting. The bayonet bracket has bearings that support the drive. Embodiments of mixing means incorporating an impeller shaft are described in U.S. Pat. Nos. 8,556,111, 8,556,497, 8,840,299 and 8,999,702.

### Multilayer Film

The body of the mixing bag comprises a flexible, water impermeable multilayer film or sheet having a thickness in a range from about 0.1 mm to about 5 mm, or preferably from about 0.2 mm to about 2 mm. Other thicknesses may also be suitable. Alternatively, as described below, the multilayer film may comprise a film and a substrate. Desirably, the multilayer film is approved for direct contact with living cells and can maintain the sterility of a solution. In such an embodiment, the multilayer film may also be sterilized, for example by radiation, ethylene oxide, or steam treatment.

Preferably, the multilayer film comprises at least two or three categorical layers including an internal contact layer that provides the inside surface of the SUB, an external layer that provides the outside surface of the SUB, and an optional gas barrier layer that is positioned between the internal contact layer and the external layer. One example of a suitable extruded material comprises a polyester elastomer outer layer, a low-density polyethylene contact layer, and an EVOH barrier layer disposed between the internal contact layer and the external layer.

The multilayer film may comprise additional layers, for example structural layers, bulking layers, or adhesion layers that are also positioned between the internal contact layer and the external layer. The multilayer film may be further laminated to an oriented film. When included, the oriented film may provide support and protection from puncture, abrasion or other abuse for the bioreactor mixing bag or SUB during its preparation and use.

### The Outside Surface Layer

The outside surface layer or external layer of the multilayer film provides the exterior surface layer of the bag and is the layer farthest from the enclosed contents. When prepared as a tubular blown film, the outside surface layer may or may not be the outermost layer of the multilayer tubular film, since the tubular film may be slit open to provide a generally planar film for use in fabricating the single-use bag.

The outside surface layer may comprise polyester, polyimide (PA), polystyrene (PS), polycarbonate (PC), poly(methyl methacrylate) (PMMA), cyclic olefin copolymer (COC), polypropylene (PP), polyethylene (PE) or a combination of two or more of these polymers.

In some embodiments, the external layer of the film comprises polypropylene or polyethylene, such as high density polyethylene (HDPE), low density polyethylene (LDPE) or linear low density polyethylene (LLDPE).

Polyethylenes are preferably selected from homopolymers and copolymers of ethylene. Various types of polyethylene homopolymers may be used in the external layer; for example, ultralow density polyethylene (ULDPE), very low density polyethylene (VLDPE), low density polyethylene (LDPE), linear low density polyethylene (LLDPE), high density polyethylene (HDPE), or metallocene polyethylene (mPE). Unless specified, the term “polyethylene” as used herein refers to polyethylene homopolymers and copolymers and to blends of polyethylene with other polymers, said blends comprising polyethylene as the major component.

Polyethylene may be made by any available process known in the art including high pressure gas, low pressure gas, solution and slurry processes employing conventional Ziegler-Natta, metallocene, and late transition metal complex catalyst systems.

Preferably, the polyethylene copolymer is an ethylene α-olefin copolymer wherein the ethylene copolymer may be an ethylene α-olefin copolymer which comprises ethylene and an α-olefin of three to twenty carbon atoms such as propylene, butene, hexene and octene, preferably of four to eight carbon atoms, such as butene, hexene and octene.

The density of the ethylene α-olefin copolymers ranges from 0.86 g/cm³ to 0.925 g/cm³, 0.86 g/cm³ to 0.91 g/cm³, 0.86 g/cm³ to 0.9 g/cm³, 0.860 g/cm³ to 0.89 g/cm³, 0.860 g/cm³ to 0.88 g/cm³, or 0.88 g/cm³ to 0.905 g/cm³. Resins made by Ziegler-Natta type catalysis and by metallocene or single site catalysis are included provided they fall within the density ranges so described. The metallocene or single site resins useful herein are (i) those which have an I-10/I-2 ratio of less than 5.63 and an Mw/Mn (polydispersity) of greater than (I-10/I-2)−4.63, and (ii) those based which have an I-10/I-2 ratio of equal to or greater than 5.63 and a polydispersity equal to or less than (I-10/I-2)−4.63. Preferably the metallocene resins of group (ii) may have a polydispersity of greater than 1.5 but less than or equal to (I-10/I-2)−4.63. Suitable conditions and catalysts which can produce substantially linear metallocene resins are described in U.S. Pat. No. 5,278,272. The reference gives full descriptions of the measurement of the well-known rheological parameters I-10 and 1-2, which are flow values under different loads and hence shear conditions. It also provides details of measurements of the well-known Mw/Mn ratio determination, as determined by gel-permeation chromatography.

Notably, the external layer may comprise the same low density polyethylene described below for the inside contact layer of the bag. To protect the sealant side of the film, which will become the inside contact layer, from exposure to other polymers in the roll and their extractable materials, an additional layer of the LDPE can be added to the outside of the film as a capping layer with an appropriate tie layer if needed. In this fashion, the sealant side contacts the same polymer only, thus protecting the inside contact layer from possible contamination.

Polypropylenes include homopolymers, random copolymers, block copolymers, terpolymers of propylene, or combinations or two or more thereof. Copolymers of propylene include copolymers of propylene with other olefin such as ethylene, 1-butene, 2-butene and the various pentene isomers, etc. and preferably copolymers of propylene with ethylene, wherein propylene is the major comonomer. Terpolymers of propylene include copolymers of propylene with ethylene and one other olefin. Random copolymers (statistical copolymers) have propylene and the comonomer(s) randomly distributed throughout the polymeric chain in ratios corresponding to the feed ratio of the propylene to the comonomer(s). Block copolymers are made up of chain segments consisting of propylene homopolymer and of chain segments consisting of, for example, random copolymers of propylene and ethylene.

Polypropylene homopolymers or random copolymers can be manufactured by any known process (e.g., using Ziegler-Natta catalyst, based on organometallic compounds or on solids containing titanium trichloride). Block copolymers can be manufactured similarly, except that propylene is generally first polymerized by itself in a first stage and propylene and additional comonomers such as ethylene are then polymerized, in a second stage, in the presence of the polymer obtained during the first.

Alternatively, the external layer comprises polyester, such as polyethylene terephthalate (PET).

Alternatively, the external layer comprises polyamide. Polyamides (e.g. nylon) suitable for use are generally prepared by polymerization of lactams or amino acids (e.g. nylon 6 or nylon 11), or by condensation of diamines such as hexamethylene diamine with dibasic acids such as succinic, adipic, or sebacic acid. The polyamides may also include copolymerized units of additional comonomers to form terpolymers or higher order polymers. The polyamide can include nylon 6, nylon 9, nylon 10, nylon 11, nylon 12, nylon 6,6, nylon 6,10, nylon 6,12, nylon 61, nylon 6T, nylon 6.9, nylon 12,12, copolymers thereof and blends of amorphous and semicrystalline polyamides. As used herein the term polyamide also includes polyamide nano-composites such as those available commercially under the tradename AEGIS polyamides from Honeywell International Inc. or IMPERM polyamide (nylon MXD6) from Mitsubishi Gas Chemical Company.

Preferred polyamides include polyepsiloncaprolactam (nylon 6); polyhexamethylene adipamide (nylon 6,6); nylon 11; nylon 12, nylon 12,12 and copolymers and terpolymers such as nylon 6/66; nylon 6,10; nylon 6,12; nylon 6,6/12; nylon 6/6, and nylon 6/6T, or blends thereof. More preferred polyamides are polyepsiloncaprolactam (nylon 6), polyhexamethylene adipamide (nylon 6,6), and nylon 6/66; most preferred is nylon 6. Although these polyamides are preferred polyamides, other polyamides, such as amorphous polyamides, are also suitable for use. Amorphous polyamides include amorphous nylon 61,6T available from E. I. du Pont de Nemours and Company under the tradename SELAR® PA. Other amorphous polyamides include those described in U.S. Pat. Nos. 5,053,259; 5,344,679 and 5,480,945. Additional useful polyamides include those described in U.S. Pat. Nos. 5,408,000; 4,174,358; 3,393,210; 2,512,606; 2,312,966 and 2,241,322.

The external layer may also comprise thermoplastic elastomers. Thermoplastic elastomers possess the ability to be stretched to moderate elongations and, upon the removal of stress, return to something close to its original shape and absence of creep. They are processable as a melt at elevated temperature, allowing them to be coextruded with other layer components.

Thermoplastic elastomers include styrenic block copolymers (TPE-s), polyolefin blends (TPE-o), elastomeric alloys (TPE-v or TPV), thermoplastic polyurethanes (TPU), thermoplastic copolyesters and thermoplastic polyamides. Examples of TPE products that come from the block copolymers group include Santoprene™ (ExxonMobil), Termoton™ by Termopol Polimer, Arnitel® (DSM), Solprene™ (Dynasol), Engage™ (Dow Chemical), copolyetheresters (Hytrel®, DuPont), (Ecdel™, Eastman), (Arnitel™, DSM Engineering), copolyesteramides (Pebax™), Dryflex™ and Mediprene™ (ELASTO), Kraton™ (Kraton Polymers), and Pibiflex™. Examples of TPV elastomers include FORPRENE™ and TERMOTON-V™. Examples of Styrenic block copolymers (TPE-s) are SOFPRENE™ (SBS) and LAPRENE™ (SEBS). Of note are copolyetheresters. Thermoplastic elastomers can provide toughness and elasticity to the overall structure.

The external layer may also comprise ionomers. The ionomers are produced from the parent acid copolymers (described below), wherein from about 10 to about 70%, or from about 30 to about 60%, of the total carboxylic acid groups of the parent acid copolymers, as calculated for the non-neutralized parent acid copolymers, are neutralized to form carboxylic acid salts with one or more alkali metal, transition metal, or alkaline earth metal cations such as for example from sodium, zinc, lithium, magnesium, and calcium; and more preferably zinc or sodium. Thus, a preferred ionomer may be chosen among E/X/Y copolymers where E is ethylene, X is (meth)acrylic acid comprising from 3 to 19, 22 or 25 weight % of the parent acid copolymer, preferably methacrylic acid, partially neutralized to salts of zinc, magnesium or sodium cations, and Y is an alkyl (meth)acrylate present in an amount of from 0 to 30 weight % of the parent acid copolymer, such as 3 to 25 weight %. Preferred alkyl (meth)acrylates include methyl acrylate, ethyl acrylate and butyl acrylate. Ionomers wherein the cations of the carboxylate salts consist essentially of sodium or zinc cations are notable. The parent acid copolymers may be neutralized using methods disclosed in, for example, U.S. Pat. No. 3,404,134.

Preferably, the ionomers used herein have a melt flow rate (MIR) of at least 0.5 gram/10 min, such as about 0.8 to about 20 grams/10 min as measured by ASTM D1238 at 190° C. using a 2160 g load. More preferably, the ionomer composition has a MFR of about 1 to about 10 grams/10 min, and most preferably has a MFR of about 1 to about 5 grams/10 min.

Blends comprising two or more ionomers may be used, provided that the aggregate components and properties of the blend fall within the limits described above for the ionomers.

Ionomers useful in the external layer layer or the structure layer (see below) are commercially available from E. I. du Pont de Nemours and Company, Wilmington, Del. (DuPont) under the Surlyn® tradename.

In some embodiments described in more detail below, a multilayer film as described herein may be adhered, for example by lamination, to a substrate comprising a monoaxially or biaxially oriented film, that serves as the external layer for material used in the single-use bag. Alternatively, the multilayer film structure may be extrusion coated onto the substrate. The monoaxially or biaxially oriented film may optionally be coated with barrier-enhancing agents.

### Gas Barrier Layer

The multilayer films preferably also comprise a gas barrier layer. The term “gas barrier layer” as used herein denotes a film layer that allows transmission through the film of less than 1000 cc of gas, such as oxygen, per square meter of film per 24 hour period at 1 atmosphere and at a temperature of 73° F. (23° C.) at 50% relative humidity. Preferably the barrier layer provides for oxygen transmission below 500, below 100, below 50, below 30 or below 15 cc/m²-day for the multilayer films. When factored for thickness, the films preferably have oxygen permeation levels of less than 40 or less than 30 cc-mil/m²-day. Other polymers may be present as additional components in the barrier layer so long as they do not raise the permeability of the barrier layer above the limit defined above.

Suitable barrier layers may be chosen from layers comprising ethylene vinyl alcohol copolymer, polyamide, polyvinyl alcohol (PVOH), polyvinylidene chloride (PVDC), poly chlorotrifluoroethylene (PCTFE), polyvinyl acetate, or blends thereof with polyethylene, polyvinyl alcohol, or polyamide. In some embodiments, the gas barrier layer is positioned between the external layer and the inside contact layer. In other embodiments, the composition of the gas barrier layer may be suitable for the external layer, so a single layer can be used to serve as both the external layer and the barrier layer.

Oriented films, metallized films or films coated SiOₓ or Al₂O₃ or PVDC may also provide gas barrier properties. Such films would be generally preformed as a substrate to which the other layers are applied, as described in more detail below.

The gas barrier layer of the multilayer films preferably comprises ethylene vinyl alcohol polymers and mixtures thereof. Unless specified, the term “EVOH” is to be understood both as ethylene vinyl alcohol polymers and blends of ethylene vinyl alcohol polymers with other polymers.

EVOH polymers generally have an ethylene content of between about 15 mole % to about 60 mole %, more preferably between about 20 to about 50 mole %. The density of commercially available EVOH generally ranges from between about 1.12 g/cm³ to about 1.20 gm/cm³, the polymers having a melting temperature ranging from between about 142° C. and 191° C. EVOH polymers can be prepared by well-known techniques or can be obtained from commercial sources. EVOH copolymers may be prepared by saponifying or hydrolyzing ethylene vinyl acetate copolymers. Thus EVOH may also be known as hydrolyzed ethylene vinyl acetate (HEVA) copolymer. The degree of hydrolysis is preferably from about 50 to 100 mole %, more preferably from about 85 to 100 mole %. In addition, the weight average molecular weight, M_{w}, of the EVOH component useful in the laminates of the invention, calculated from the degree of polymerization and the molecular weight of the repeating unit, may be within the range of about 5,000 Daltons to about 300,000 Daltons with about 60,000 Daltons being most preferred.

Suitable EVOH polymers may be obtained from Eval Company of America or Kuraray Company of Japan under the tradename EVAL™. EVOH is also available under the tradename SOARNOL™ from Noltex L.L.C. Examples of such EVOH resins include EVAL® grades F101, F171, E105, J102, and SOARNOL™ grades DT2903, DC3203 and ET3803. Of special note are EVOH resins sold under the tradename EVAL® SP obtained from Eval Company of America or Kuraray Company of Japan that may be useful as components in the films of the invention. EVAL™ SP is a type of EVOH that exhibits enhanced plasticity and that is suited for use in packaging applications including shrink film, polyethylene terephthalate (PET)-type barrier bottles and deep-draw cups and trays. Examples of such EVOH resins include EVAL™ SP grades 521, 292 and 482. The EVAL SP grades of EVOH are easier to orient than the conventional EVAL resins. These EVOH polymers are a preferred class for use in the multilayer film compositions described herein. Without being bound to theory, it is believed that the enhanced orientability of these resins derives from their chemical structure, in particular the level of head to head adjacent hydroxyl groups in the EVOH polymer chain. By head to head adjacent hydroxyl groups is meant 1,2-glycol structural units.

It has been found that EVOH polymers having a relatively high level of 1,2-glycol units in the EVOH polymer chain are particularly suited for use in multilayer film. For example, about 2 to about 8 mol % 1,2-glycol structural units, preferably about 2.8 to about 5.2 mol % 1,2-glycol units may be present in the EVOH polymer chain.

Such polymers can be produced by increasing the amount of adjacent copolymerized units of vinyl acetate produced during polymerization of ethylene and vinyl acetate above the level generally used. When such polymers are hydrolyzed to form EVOH, an increased amount of head-to-head vinyl alcohol adjacency, that is, an increased amount of the 1,2-glycol structure result. It has been reported in the case of polyvinyl alcohol that the presence of the 1,2-glycol structure in polyvinyl alcohol can influence the degree of crystallinity obtained in these alcohols and thereby the tensile strength. See, for example F. L. Marten & C. W. Zvanut, Chapter 2 Manufacture of Polyvinyl Acetate for Polyvinyl Alcohol, Polyvinyl Alcohol Developments (C. A. Finch ed.) John Wiley, New York 1992.

The more orientable grades of EVOH will generally have lower yield strength, lower tensile strength and lower rates of strain hardening than other EVOH polymers of equivalent ethylene content, as measured by mole % ethylene.

The EVOH composition may optionally be modified by including additional polymeric materials selected from the group consisting of polyamides, including amorphous polyamides such as 61/6T copolyamides, MXD6, polyvinyl acetate (PVA), ionomers, and ethylene polymers and mixtures thereof. These modifying polymers may be present in amounts up to 30 weight % of the EVOH composition.

In a preferred embodiment, the coextruded multilayer structure may comprise a layer of EVOH sandwiched between two layers of polyamide, one on each side of the EVOH layer. This leads to a maximum possible oxygen barrier and at the same time ensures excellent embedding and stabilization of the EVOH layer between the two polyamide layers as carrier layers. For example, a multilayer film useful herein could comprise a polyamide layer that functions as an external layer and a layer that sandwiches the EVOH layer: PA/EVOH/PA/tie/LDPE*, wherein LDPE* indicates an antioxidant-five polyethylene as described herein.

Alternatively, the polyamide layers may comprise blends of PA and EVOH, or PA and PVA or PA and MXD6, respectively.

As the barrier properties of EVOH may be influenced negatively by humidity, it is potentially beneficial that a moisture barrier layer be positioned between the EVOH layer and the moist contents of the package. The inside contact layer described herein may be a sufficient moisture barrier. Also, using desiccant in polymer layers between the moisture source and the EVOH layers may be an effective way to reduce the impact of moisture on the barrier of EVOH.

### Structure Layer

The structure layer provides desired mechanical properties for the multilayer film and may also provide bulking properties. The structure layer(s) when present may also desirably provide moisture barrier properties.

Polyethylenes such as linear low density polyethylene or metallocene polyethylene, ionomers and thermoplastic elastomers described above may also be suitable as structure layer compositions. Whether used as an external layer or a structure layer, they provide toughness or improved impact resistance to the multilayer film.

The structure layer may comprise an ethylene copolymer. The term “ethylene copolymer” refers to a polymer comprising copolymerized units derived from ethylene and at least one additional monomer, especially a polar comonomer such as vinyl acetate, alkyl (meth)acrylate, (meth)acrylic acid or glycidyl methacrylate. The ethylene copolymer may be chosen among ethylene vinyl acetate copolymers, ethylene alkyl (meth)acrylate copolymers, ethylene alkyl (meth)acrylic acid copolymers or ionomers thereof, or combinations of two or more thereof.

In the case where the structure layer comprises an ethylene vinyl acetate (EVA) copolymer, the relative amount of copolymerized vinyl acetate units may be of from 2 to 40 weight %, preferably from 10 to 40 weight %, the weight percentage being based on the total weight of the ethylene vinyl acetate copolymer. A mixture of two or more different ethylene vinyl acetate copolymers may be used as components of the structure layer in place of a single copolymer.

The structure layer may comprise an ethylene alkyl (meth)acrylate copolymer. Ethylene alkyl (meth)acrylate copolymers are thermoplastic ethylene copolymers derived from the copolymerization of ethylene comonomer and at least one alkyl (meth)acrylate comonomer, wherein the alkyl group contains from one to ten carbon atoms and preferably from one to four carbon atoms. The relative amount of copolymerized alkyl (meth)acrylate units may be of from 0.1 to 45 weight %, preferably from 5 to 35 weight % and still more preferably from 8 to 28 weight %, the weight percentage being based on the total weight of the ethylene alkyl (meth)acrylate copolymer. Preferably, the ethylene alkyl (meth)acrylate copolymer is an ethylene methyl acrylate, ethylene ethyl acrylate, or ethylene butyl acrylate copolymer.

The structure layer may comprise an ethylene alkyl (meth)acrylic acid copolymer, or preferably an ionomer thereof.

The ethylene alkyl (meth)acrylic acid copolymer can be an E/X/Y copolymer where E represents copolymerized units of ethylene, X represents copolymerized units of a C₃ to C₈ α,β-ethylenically unsaturated carboxylic acid, and Y represents copolymerized units of an optional comonomer selected from alkyl acrylate and alkyl methacrylate.

The C₃ to C₈ α,β-ethylenically unsaturated carboxylic acid may be present of from 2 weight % to 30 weight %, preferably of from 5 weight % to 20 weight %, and most preferably of from 12 weight % to 19 weight %, based on the total weight of the ionomer. Suitable C₃ to C₈ α,β-ethylenically unsaturated carboxylic acids may be chosen among methacrylic acid and acrylic acid, with methacrylic acid being preferred.

The alkyl acrylate or alkyl methacrylate comonomer may optionally be present in an amount from 0.1 weight % to 40 weight %, or from 5 weight % to 35 weight %, or from 8 to 30 weight %, or from about 18 to about 30 weight %, or from about 19 to about 25 weight %, or from about 19 to about 23 weight % of the total weight of the E/X/Y copolymer.

Preferably, the alpha, beta-ethylenically unsaturated carboxylic acid is methacrylic acid. Of note are acid copolymers consisting essentially of copolymerized units of ethylene and copolymerized units of the alpha, beta-ethylenically unsaturated carboxylic acid and 0 weight % of additional comonomers; that is, dipolymers of ethylene and the alpha, beta-ethylenically unsaturated carboxylic acid. Preferred acid copolymers are ethylene methacrylic acid dipolymers.

The ethylene acid copolymers used herein may be polymerized as disclosed in U.S. Pat. Nos. 3,404,134; 5,028,674; 6,500,888; and 6,518,365.

### The Inside Contact Layer

The inside surface layer, or contact layer, is the interior surface layer of a single-use bag prepared from the multilayer film and is in contact with the bag's contents. As noted above, the inside contact layer comprises polyethylene that does not contain antioxidants or other additives that may be extracted into the contents of the SUB.

The inside contact layer also provides a means for sealing or closing the SUB, for example by heat sealing one portion of the inside contact layer to another portion. Alternatively, one portion of the inside contact layer may be heat sealed to the surface of another part of the SUB, such as a port or another fitment. Among other factors, the composition of the inside contact layer is selected to influence the sealing capability of the inside surface layer, i.e., such that a high sealing bond strength may be achieved.

The inside contact layer comprises low density polyethylene (0.88 g/cm³ to 0.925 g/cm³) made in an autoclave free-radical polymerization process within the two-phase region of the autoclave. The molecular weight distribution of the polymer made in two-phase conditions is different than the molecular weight distribution of single-phase LDPE, giving higher molecular weight (Mn, Mz, Mv, and Mw) and a narrower polydispersity index for a given melt index (MI). Preferably, the LDPE has a melt index from 0.8 to 2.5 g/10 min (measured according to ASTM D1238 at 190° C. using a 2160 g load) with a weight average molecular weight (Mw) greater than 200,000 Da, a z-average molecular weight (Mw) greater than 1,500,000 Da, and a polydispersity of 6 to 8. Similar single-phase LDPE having a melt index from 0.9 to 2.0 g/10 min has Mw less than 180,000 Da, an Mz less than 1,000,000 Da, and a polydispersity of 7 to 13.

The two-phase polymerization process produces LDPE polymer that may have higher environmental stress cracking resistance (ESCR, ASTM D1693), up to twice as high for a given MI, compared to LDPE produced in a single-phase process. Moreover, LDPE produced by a two-phase process may exhibit lower melt swell (about 10% less) than LDPE produced in a single-phase process.

Notably, the contact layer comprises a low density polyethylene that does not have added antioxidants, especially phosphite antioxidants, antistatic agents, brighteners, cure reagents, lubricants, thermal stabilizers, plasticizers or processing aids such as slip or antiblock additives. Low-density polyethylenes made in a two-phase process without added antioxidants, especially phosphite antioxidants, antistatic agents, brighteners, cure reagents, lubricants, thermal stabilizers, plasticizers or processing aids, such as slip or antiblock additives, are available commercially as DuPont™ 20 Series DPE-20 and DPE2020T from E. I. du Pont de Nemours, Wilmington, Del. (DuPont). These polymers are produced without processing aids. DPE-20 yields a low extractable profile because of the absence of antioxidant in its composition and because of its lower level of small molecules, shown by the Mn, Mw, Mz and Mv.

The compositions of the structure layer and the contact layer may provide a desirable water vapor barrier to protect the gas barrier layer from reduced efficiency due to the presence of vapor that may permeate through the film from the contents of the bag to the EVOH layer.

### Adhesion Layers

In addition, the multilayer film may comprise one or more additional layers to serve as adhesion layers between functional layers to improve interlayer adhesion and to prevent delamination of the layers. For example, such adhesion layers may be positioned between the external layer and the gas barrier layer, between the gas barrier and the structure layer, or between the structure layer and the contact layer. In some embodiments, the contact layer comprises polyethylene and the structure layer may comprise an ionomer. In such embodiments, an adhesion layer may be necessary to provide sufficient interlayer adhesion.

The adhesion layer(s) are compositionally distinct from the structure layer and from the contact layer. The term “compositionally distinct”, as used herein, refers to two compositions in which the number of components, the ratio of components, or the chemical structure (for example, comonomer ratio of polymeric components having the same comonomers) of one or more of the components are different.

Adhesion layer compositions described in U.S. Pat. Nos. 6,545,091, 5,217,812, 5,053,457, 6,166,142, 6,210,765 and U.S. Patent Application Publication 2007/0172614, for example, are suitable for use in the SUBs. Preferred adhesion layers comprise a multicomponent composition comprising (1) a functionalized polymer, (2) an ethylene polymer or copolymer or propylene polymer or copolymer, 3) optionally a tackifier, and 4) optionally a toughening polymer as described above. These multicomponent compositions are particularly suitable for use as an adhesion or “tie” layer in multilayer films.

The functionalized polymers useful as component 1) of the preferred multicomponent adhesion composition include anhydride-modified polymers and copolymers comprising copolymerized units of ethylene and a comonomer selected from the group consisting of C₄-C₈ unsaturated acids having at least two carboxylic acid groups, and cyclic anhydrides, monoesters and diesters of such acids. Mixtures of these components are also useful. The ethylene polymers or copolymers useful as component 2) of the multicomponent adhesion composition comprise at least one ethylene polymer or copolymer that is chemically distinct from the functionalized polymer that is the component 1) of the multicomponent adhesion composition. More specifically, a) the ethylene copolymer of the component 2) of the multicomponent adhesion composition comprises at least one species of copolymerized monomer that is not present as a comonomer in the functionalized polymer component; b) the functionalized polymer component comprises at least one species of copolymerized monomer that is not present in the ethylene copolymer of the component 2); or c) the ethylene copolymer that is the component 2) of the adhesion is not an anhydride-grafted or functionalized ethylene copolymer as defined above. Thus, the first and second polymers are different in chemical structure and are distinct polymer species.

The functionalized polymer may be a modified copolymer, meaning that the copolymer is grafted or copolymerized with organic functionalities. Modified polymers for use in the tie layer may be modified with acid, anhydride or epoxide functionalities. Examples of the acids and anhydrides used to modify polymers, which may be mono-, di- or polycarboxylic acids are acrylic acid, methacrylic acid, maleic acid, maleic acid monoethylester, fumaric acid, fumaric acid, itaconic acid, crotonic acid, itaconic anhydride, maleic anhydride and substituted maleic anhydride, e.g. dimethyl maleic anhydride or citrotonic anhydride, nadic anhydride, nadic methyl anhydride, and tetrahydrophthalic anhydride, or combinations of two or more thereof, maleic anhydride being preferred.

In the case where the one or more olefin homopolymers or copolymers are acid-modified, the content of grafted or directly co-polymerized acid may range from from 0.05 to 25 weight %, the weight percentage being based on the total weight of the modified polymer.

Modified polymers that are preferred for use as functionalized polymer components of the multicomponent adhesion composition are anhydride-grafted homopolymers or copolymers.

When an anhydride-modified polymer is used, it may contain from 0.03 to 10 weight %, 0.05 to 5 weight %, or 0.05 to 3% of a grafted anhydride, the weight percentage being based on the total weight of the modified polymer. These include polymers that have been grafted with from 0.1 to 10 weight % of an unsaturated dicarboxylic acid anhydride, preferably maleic anhydride. Generally, the grafted polymers are polyolefins, for example grafted polyethylene, grafted polypropylene, grafted EVA copolymers, grafted ethylene alkyl acrylate copolymers and grafted ethylene alkyl methacrylate copolymers, each grafted with from 0.1 to 10 weight % of an unsaturated dicarboxylic acid anhydride. Specific examples of suitable anhydride-modified polymers are disclosed in U.S. Patent Application Publication 2007/0172614.

The functionalized polymer may also be an ethylene copolymer comprising copolymerized units of ethylene and a comonomer selected from the group consisting of C₄-C₈ unsaturated anhydrides, monoesters of C₄-C₈ unsaturated acids having at least two carboxylic acid groups, diesters of C₄-C₈ unsaturated acids having at least two carboxylic acid groups and mixtures of such copolymers. The ethylene copolymer may comprise from about 3 to about 25 weight % of copolymerized units of the comonomer. The copolymer may be a dipolymer or a higher order copolymer, such as a terpolymer or tetrapolymer. The copolymers are preferably random copolymers. Examples of suitable comonomers of the ethylene copolymer include unsaturated anhydrides such as maleic anhydride and itaconic anhydride; C₁-C₂₀ alkyl monoesters of butenedioic acids (e.g. maleic acid, fumaric acid, itaconic acid and citraconic acid), including methyl hydrogen maleate, ethyl hydrogen maleate, propyl hydrogen fumarate, and 2-ethylhexyl hydrogen fumarate; C₁-C₂₀ alkyl diesters of butenedioic acids such as dimethylmaleate, diethylmaleate, and dibutylcitraconate, dioctylmaleate, and di-2-ethylhexylfumarate. These functionalized polymer components of the adhesion composition are ethylene copolymers obtained by a process of high-pressure free radical random copolymerization, rather than graft copolymers. The monomer units are incorporated into the polymer backbone or chain and are not incorporated to an appreciable extent as pendant groups onto a previously formed polymer backbone.

Examples of epoxides used to modify polymers are unsaturated epoxides comprising from four to eleven carbon atoms, such as glycidyl (meth)acrylate, allyl glycidyl ether, vinyl glycidyl ether and glycidyl itaconate, glycidyl (meth)acrylates being particularly preferred.

Epoxide-modified ethylene copolymers preferably contain from 0.03 to 15 weight %, 0.03 to 10 weight %, 0.05 to 5 weight %, or 0.05 to 3% of an epoxide, the weight percentage being based on the total weight of the modified ethylene copolymer. Preferably, epoxides used to modify ethylene copolymers are glycidyl (meth)acrylates. The ethylene/glycidyl (meth)acrylate copolymer may further contain copolymerized units of an alkyl (meth)acrylate having from one to six carbon atoms. Representative alkyl (meth)acrylates include methyl (meth)acrylate, ethyl (meth)acrylate, propyl (meth)acrylate, butyl (meth)acrylate, isobutyl (meth)acrylate, hexyl (meth)acrylate, or combinations of two or more thereof. Of note are ethyl acrylate and butyl acrylate. Preferably, modified ethylene copolymers comprised in the tie layer are modified with acid, anhydride or glycidyl (meth)acrylate functionalities.

The ethylene copolymers suitable for use in adhesion layers of the coextruded multilayer film structure can be produced by any means known to one skilled in the art using either autoclave or tubular reactors (e.g. U.S. Pat. Nos. 3,404,134, 5,028,674, 6,500,888, 3,350,372, and 3,756,996).

Preferably, each adhesion layer independently comprises a functionalized polymer comprising grafted polyethylene, grafted EVA copolymers, grafted ethylene alkyl acrylate copolymers or grafted ethylene alkyl methacrylate copolymers, each grafted with from 0.1 to 10 weight % of an unsaturated dicarboxylic acid anhydride; or copolymers comprising copolymerized units of ethylene and a comonomer selected from the group consisting of C₄-C₈ unsaturated acids having at least two carboxylic acid groups, and cyclic anhydrides, monoesters and diesters of such acids.

Compositions comprising olefin polymers and modified polymers thereof are commercially available under the trademarks APPEEL®, BYNEL®, ELVALOY®AC, and ELVAX® from DuPont.

A second component of the preferred adhesion composition may be at least one ethylene polymer or copolymer compositionally distinct from the first functionalized polymer component. Ethylene polymers or copolymers used as the second component of the adhesion composition may be polyethylene homopolymers, copolymers of ethylene and alpha-olefins, including copolymers with propylene and other alpha-olefins. Ethylene polymers or copolymers suitable for use as the second component include high density polyethylenes, low density polyethylenes, very low density polyethylenes (VLDPE), linear low density polyethylenes, and copolymers of ethylene and alpha-olefin monomers prepared in the presence of metallocene catalysts, single site catalysts and constrained geometry catalysts (herein referred to as metallocene polyethylenes, or MPE). Suitable ethylene copolymers and methods for their preparation are disclosed in U.S. Patent Application Publication 2007/0172614. The ethylene copolymer used as the second component of the adhesion composition may also comprise copolymerized units of ethylene and a polar comonomer such as vinyl acetate, alkyl acrylates, alkyl methacrylates and mixtures thereof. The alkyl groups will have from 1 to 10 carbon atoms. Additional comonomers may be incorporated as copolymerized units in the ethylene copolymer. Suitable copolymerizable monomers include carbon monoxide, methacrylic acid and acrylic acid. Ethylene alkyl acrylate carbon monoxide terpolymers are examples of such compositions, including ethylene n-butyl acrylate carbon monoxide terpolymers.

The ethylene copolymer of the second component may also be an ethylene alkyl acrylate or ethylene alkyl methacrylate copolymer. Alkyl acrylates and alkyl methacrylates may have alkyl groups of 1 to 10 carbon atoms, for example methyl, ethyl or butyl groups. The relative amount of the alkyl acrylate or alkyl methacrylate comonomer units in the copolymers can vary broadly from a few weight % to as much as 45 weight %, based on the weight of the copolymer. Mixtures of ethylene alkyl acrylate or alkyl methacrylate copolymers may also be used.

The adhesion composition may also include a tackifier. The presence of tackifier facilitates bond adhesion when the film is oriented and later shrunk. The tackifier may be any suitable tackifier known generally in the art. For example, the tackifier may include types listed in U.S. Pat. No. 3,484,405. Suitable tackifiers include a variety of natural and synthetic resins and rosin materials. Tackifier resins that can be employed are liquid, semi-solid to solid, complex amorphous materials generally in the form of mixtures of organic compounds having no definite melting point and no tendency to crystallize. These include coumarone-indene resins, such as the para-coumarone-indene resins, terpene resins, including styrenated terpenes, butadiene-styrene resins having molecular weights ranging from about 500 to about 5,000, polybutadiene resins having molecular weights ranging from about 500 to about 5,000, hydrocarbon resins produced by catalytic polymerization of fractions obtained in the refining of petroleum, having a molecular weight range of about 500 to about 5,000, polybutenes obtained from the polymerization of isobutylene, hydrogenated hydrocarbon resins, rosin materials, low molecular weight styrene hard resins or disproportionated pentaerythritol esters, aromatic tackifiers, including thermoplastic hydrocarbon resins derived from styrene, alpha-methylstyrene, or vinyltoluene, and polymers, copolymers and terpolymers thereof, terpenes, terpene phenolics, modified terpenes, and combinations thereof. These latter materials may be further hydrogenated in part or in entirety to produce alicyclic tackifiers. A more comprehensive listing of tackifiers that can be employed in this invention is provided in TAPPI CA Report #55, Technical Association of the Pulp and Paper Industry, 1975, pp 13-20, which lists over 200 commercially available tackifier resins.

The thickness of each adhesion layer of the multilayer structure may be independently between 1 and 100 μm, 5 and 50 μm, or 5 to 30 μm.

The various layer compositions of the coextruded multilayer film structure may further comprise modifiers and other additives except where noted for the contact layer composition, including without limitation, plasticizers, impact modifiers, stabilizers including viscosity stabilizers and hydrolytic stabilizers, lubricants, antioxidants, UV light stabilizers, antifog agents, antistatic agents, dyes, pigments or other coloring agents, fillers, flame retardant agents, reinforcing agents, foaming and blowing agents and processing aids known in the polymer compounding art like for example antiblock agents and release agents.

These additives may be present in each layer composition independently in amounts of up to 20 weight %, preferably from 0.01 to 7 weight %, and more preferably from 0.01 to 5 weight %, the weight percentage being based on the total weight of the composition. Desirably, in layers other than the contact layer such additives are included at the lowest level required to perform their function, with minimal chance of migration out of the layers into the contact layer.

The multilayer film structure may be prepared by coextrusion of the layers in blown film or cast film processes well known in the art. It may also be prepared by (co)extrusion coating of one or more layers in molten form onto a preformed film comprising the other layers of the multilayer structure. It may also be prepared by (co)extrusion wherein one or more inner layers are laid down in molten form between two preformed films that comprise the other layers of the multilayer structure.

### Substrate Layer

In another embodiment according to the invention, the multilayer film structure described above is part of a structural component of the bag that has a carrier material or substrate for the multilayer structure. In such embodiments the multilayer structure is effectively protected from mechanical wear, abrasion, puncture or other abuse and it can therefore ensure the desired water and barrier properties over an extended period of time during its use. The substrate also provides structural support for the multilayer structure.

More specifically the multilayer film structure described above is a monolithic or continuous membrane on the surface of the substrate so that it can maintain the continuity required to form a bag that can contain fluids. However, the substrate need not be continuous. For example, cut-outs, holes, or other open areas may be included in the substrate so that it does not fully overlay the film structure. Such discontinuities in the substrate may allow for areas of the film to remain uncovered to allow for transparent viewing areas into the interior of the bag, to facilitate insertion of ports, fitment, or attachments, to provide lightweighting of the bag structure in areas not needing the additional support or protection provided by the substrate, or for any other reason.

More specifically, the mixing bag may comprise a combination of multilayer film and the multilayer structure-substrate material. For example, one or more panels of the mixing bag may comprise the multilayer structure-substrate material and one or more panels of the mixing bag may comprise the multilayer film. The film may be used in portions of the mixing bag where it is desirable to be able to view the contents of the mixing bag in use (such as a top panel), while the multilayer structure-substrate material may be used in portions of the bag (such as the bottom) needing more support or protection against mechanical influences such as punctures or abrasion.

The substrate may comprise monoaxially or biaxially oriented films such as nylon, polyester, EVOH or polypropylene, optionally coated with a barrier-enhancing agent such as SiOₓ or Al₂O₃ or polyvinylidene chloride (PVDC). Of note is biaxially oriented nylon, optionally coated with PVDC or metallized.

The multilayer structure may be applied to any of these carrier materials as a film or membrane or as a coating, via (co)extrusion coating, lamination, other appropriate application methods or combinations thereof.

For example, the multilayer structure is applied to the carrier material as a film, a coating or a laminated layer. Normally the carrier material is coated or laminated on one side. The coating or laminate is applied to the substrate so that the contact layer for the mixing bag is an exposed surface layer. The mixing bag is assembled so that the contact layer faces the interior of the mixing bag and the protective carrier material faces toward the side facing the exterior of the mixing bag.

Of note is an embodiment wherein the material comprising an internal contact layer described herein is applied to a film by, for example but not limitation, coextrusion coating. For example, coextrusion coating the multilayer structure onto the film can be done as follows: granulates of the composition for each of the layers are melted in single screw extruders. The molten polymers are passed through a flat die to form a molten polymer curtain wherein the compositions of the individual layers are present in laminar flow. The molten curtain drops onto the moving film substrate to be immediately pressed onto that substrate and quenched by a quench drum.

A film as described herein, comprising an internal contact layer comprising polyethylene that does not contain antioxidant, can also be laminated to a substrate comprising an oriented film by means of an adhesion layer applied in molten form to adhere the film to the substrate. The process involves laying down a molten curtain of the adhesion layer composition between the film and the oriented film substrate moving at high speeds as they come into contact with a cold (chill) roll. The melt curtain is formed by extruding the adhesion layer composition through a flat die. Compositions for the adhesion layer may be those described above, so long as they provide sufficient adhesion between the oriented film substrate and the adjacent layer of the multilayer structure. Optionally, the surfaces of the laminate to be joined may be additionally treated using plasma, corona, IR, priming, or flame to increase adhesion. Notable thermoplastic adhesion compositions comprise ethylene/vinyl acetate copolymers.

Alternatively, the substrate and a film comprising the multilayer structure can be adhered using non-thermoplastic adhesives such as water based adhesives, or solvent based single component polyurethanes. Solvent-based adhesives can be applied to the substrate by in any suitable manner known in the art, including gravure coating, roll coating, wire rod coating, dip coating, flexographic printing, spray coating and the like. Excess adhesive coating composition can be removed by squeeze rolls, doctor knives and the like, if desired. A film comprising the multilayer structure is applied over the adhesive and the solvent is removed by heating.

Hot melt adhesives may also be suitable. Hot melt adhesives are much less viscous than the thermoplastic extrudable adhesion compositions described above. Hot melt glues may be applied to the substrate by coating methods described above using liquefied hot glue. The multilayer film is laid over the hot melt adhesive and the adhesive is activated by applying heat to the overlaid webs. The method can be run as a continuous process using a combination of coating and heating such as the use of heated rollers.

In some embodiments the film may be adhered to the substrate in discontinuous fashion. For example, the adhesive may be present as a discontinuous layer between the film and the substrate, and in many cases, it may be applied as a series of adhesive dots that cover for example about 10 to about 40 percent of the substrate surface. The adhesive also may be applied selectively near the edges of the film and the substrate.

The film may also be attached to the substrate by heat sealing or high frequency (HF) welding. The laminate can be heat sealed (thermally bonded) using any known method, included heated presses and calenders and the like, or by applying heat to the layers and then subsequently pressing them together without additional heat. In each case, the softened layer or component subsequently bonds the film structure to the substrate. In either heat sealing or HF welding, the bonding of the film to the substrate may be continuous across the entire area of the film and substrate or it may be discontinuous. Discontinuous bonding may be accomplished by application of heat or HF radiation to selected portions of the area where the film overlays the substrate.

The multilayer sheet comprising a film substrate can be formed into the bag in manners similar to those described above for the multilayer film, such as by cutting the appropriately shaped panels and heat sealing the contact layer(s) of the panels together. Dielectric bonding can be effective in some circumstances, as is ultrasonic sealing.

### Preferred Multilayer Films

Preferred multilayer films for use in the single-use bag comprise at least three categorical layers including an internal contact layer that provides the inside surface of the single-use bag comprising the two-phase LDPE described above, an external layer that provides the outside surface of the mixing bag, and a gas barrier layer that is positioned between the internal contact layer and the external layer.

Preferred multilayer films comprise external layers comprising polyethylene, polypropylene, polyester, polyamide, thermoplastic elastomer or ionomer; a gas barrier layer comprising EVOH or EVOH between two layers of polyamide; and at least one additional layer including structure layer, bulking layer or adhesion layer positioned between the internal contact layer and the external layer, preferably wherein the structure provides toughening and comprises a thermoplastic elastomer, ionomer or polyethylene such as LLDPE or mPE.

Preferred multilayer structure-substrate sheets include those wherein the substrate is an oriented film, preferably oriented polyethylene terephthalate (OPET), oriented polypropylene (OPP) or biaxially oriented nylon (BON). Any of the preferred films or multilayer structure-substrate sheets used in the bag may further comprise a capping layer comprising the two-phase LDPE described above and an adhesion layer where needed on the external surface of the bag.

Representative examples of suitable multilayer films and laminates include, without limitation, those described below. In these structures, outside to inside layers of the multilayer structure as intended to be used in a single-use bag are listed in order from left to right. In the multilayer film structures, the symbol “/” represents a boundary between layers. The symbol “//” represents a boundary between a film layer and an oriented film substrate. “PE” represents any polyethylene, but may specifically refer to LDPE*. “LDPE*” refers to an antioxidant-free low density polyethylene composition used as the contact layer. “TPE” refers to a thermoplastic elastomer, preferably a copolyester ether. Where a (co)extrudable adhesion layer is present, that layer is designated as “tie.” Tie layer compositions in a structure may be the same or different, depending on the compositions of adjacent layers. Where a solvent-based adhesive or hot melt adhesive is present, that layer is designated as “adh”. Oriented films are indicated by “0” followed by a designation for the polymer of the composition. Biaxially oriented films are indicated by “BO” followed by a designation for the polymer of the composition. Oriented or biaxially oriented films optionally may be coated with barrier agents as described above. Those skilled in the art will recognize that other film structures are suitable for use in the SUB described herein. Such structures may include one or more tie or adhesion layers, comprising any adhesion composition, including the above-described preferred adhesion compositions. Each embodiment has particular advantages depending on the particular application.

| | |
|---|---|
| PE/tie/EVOH/EVA/LDPE*; | PP/tie/EVOH/EVA/LDPE*; |
| PE/tie/ionomer/tie/EVOH/tie/LDPE* | PP/tie/ionomer/tie/EVOH/tie/LDPE* |
| PE/tie/TPE/tie/EVOH/tie/LDPE* | PP/tie/TPE/tie/EVOH/tie/LDPE* |
| PE/tie/ionomer/tie/PA/EVOH tie/LDPE* | PP/tie/ionomer/tie/PA/EVOH tie/LDPE* |
| PE/tie/PA/EVOH/PA/tie/LDPE*; | PP/tie/PA/EVOH/PA/tie/LDPE*; |
| PE/tie/PA/EVOH/PA/tie/EVA/tie/LDPE*; | PP/tie/PA/EVOH/PA/tie/EVA/tie/LDPE*; |
| PET/tie/EVOH/EVA/LDPE*; | PA/tie/EVOH/EVA/LDPE*; |
| PET/tie/ionomer/tie/EVOH/tie/LDPE* | PA/tie/ionomer/tie/EVOH/tie/LDPE* |
| PET/tie/TPE/tie/EVOH/tie/LDPE* | PA/tie/TPE/tie/EVOH/tie/LDPE* |
| PET/tie/ionomer/tie/PA/EVOH tie/LDPE* | PA/tie/ionomer/tie/PA/EVOH tie/LDPE* |
| PET/tie/PA/EVOH/PA/tie/LDPE*; | PA/tie/PA/EVOH/PA/tie/LDPE*; |
| PET/tie/PA/EVOH/PA/tie/EVA/tie/LDPE*; | PA/tie/PA/EVOH/PA/tie/EVA/tie/LDPE*; |

PE/tie/PA/EVOH/PA/tie/ethylene alkyl acrylate/tie/LDPE*;
PP/tie/PA/EVOH/PA/tie/ethylene alkyl acrylate/tie/LDPE*;
PA/tie/PA/EVOH/PA/tie/ethylene alkyl acrylate/tie/LDPE*;
PET/tie/PA/EVOH/PA/tie/ethylene alkyl acrylate/tie/LDPE*;

| | |
|---|---|
| Ionomer/tie/EVOH/tie/LDPE* | TPE/tie/EVOH/tie/LDPE* |
| Ionomer/tie/PA/EVOH/PA/tie/LDPE* | TPE/tie/PA/EVOH/PA/tie/LDPE* |
| Ionomer/tie/EVOH/EVA/LDPE*; | TPE/tie/EVOH/EVA/LDPE*; |
| Ionomer/tie/PA/EVOH/PA/EVA/LDPE*; | TPE/tie/PA/EVOH/PA/EVA/LDPE*; |
| BON//tie/TPE/tie/EVOH/tie/LDPE*; | BON//tie/ionomer/tie/EVOH/tie/LDPE*; |
| BON//tie/LLDPE/tie/EVOH/tie/LDPE*; | BON//tie/mPE/tie/EVOH/tie/LDPE*; |
| BON//tie/TPE/tie/PA/EVOH/PA/tie/LDPE*; | BON//tie/mPE/tie/PA/EVOH/PA/tie/LDPE*; |
| BON//tie/LLDPE/tie/PA/EVOH/PA/tie/LDPE*; | BON//tie/ionomer/tie/PA/EVOH/PA/tie/LDPE*; |
| OPP//tie/TPE/tie/EVOH/tie/LDPE*; | OPP//tie/ionomer/tie/EVOH/tie/LDPE*; |
| OPP//tie/LLDPE/tie/EVOH/tie/LDPE*; | OPP//tie/mPE/tie/EVOH/tie/LDPE*; |
| OPP//tie/TPE/tie/PA/EVOH/PA/tie/LDPE*; | OPP//tie/mPE/tie/PA/EVOH/PA/tie/LDPE*; |
| OPP//tie/LLDPE/tie/PA/EVOH/PA/tie/LDPE*; | OPP//tie/ionomer/tie/PA/EVOH/PA/tie/LDPE*; |
| OPET//tie/TPE/tie/EVOH/tie/LDPE*; | OPET//tie/ionomer/tie/EVOH/tie/LDPE*; |
| OPET//tie/LLDPE/tie/EVOH/tie/LDPE*; | OPET//tie/mPE/tie/EVOH/tie/LDPE*; |
| OPET//tie/TPE/tie/PA/EVOH/PA/tie/LDPE*; | OPET//tie/ionomer/tie/PA/EVOH/PA/tie/LDPE*; |
| OPET//tie/LLDPE/tie/PA/EVOH/PA/tie/LDPE*; | OPET//tie/mPE/tie/PA/EVOH/PA/tie/LDPE*; |

Other representative structures include any of the structures in the preceding list wherein a capping layer comprising LDPE* and a tie layer or adhesive layer where needed is applied to the outside layer of the structure.

The flexible film described herein may also be used for single-use fluid containers, reservoirs, bags, bladders, pouches or membranes for storage, mixing, pumping, transfer or delivery of medicaments, pharmaceuticals, diluents, sera, intravenous solutions, whole blood, plasma, blood fractions and other fluids intended for treatment of humans or animals, including for example, intravenous (IV) bags, pump bladders, ostomy pouches and the like.

### Methods

The invention also provides a method for preparing a biological product, the method comprising providing a single-use bioreactor comprising the single-use flexible film bag as described herein; placing inside the compartment a culture medium and a biological sample; and allowing the biological sample to interact with the culture medium to provide a cell culture medium containing the biological product.

The method may further comprise removing at least a portion of the cell culture medium from the single-use flexible film bag; and transforming the portion of the cell culture medium to obtain the biological product.

Many cell culture processes are run in so-called batch-fed batch mode, where all cells, reactants, culture media, nutrients and the like needed to run the process are charged to the bioreactor at the beginning of the process and waste products, metabolites and other outputs are not removed until the process is completed after a period of time.

Alternatively, perfusion cell culture may be used, in which a constant flow of nutrient medium is fed into the bioreactor and metabolites are removed in a continuous process. The major advantage of the perfusion mode is high cell number and high productivity in a relatively small-size bioreactor as compared with batch/fed-batch. In order to sustain high cell number and productivity, there are needs to feed medium during the cell propagation phase and the production phase. In contrast to batch and fed-batch processes, where there is no metabolites removal, in continuous processes medium is perfused at dilution rates exceeding the cellular growth rate. For this, a good separation device is needed to retain cells in the bioreactor, including gravity-based cell settlers, spin filters, centrifuges, cross-flow filters, alternating tangential-flow filters, vortex-flow filters, acoustic settlers (sonoperfusion), and hydrocyclones.

Bioreactors designed to be run in either batch mode or perfusion mode can be prepared using the single-use bags described herein.

Finally, further provided herein is a biological product obtained by placing a culture medium and a biological sample inside the compartment of a single-use bag as described herein, and allowing the biological sample to interact with the culture medium to provide a cell culture medium containing the biological product. The biological product may be obtained by transforming at least a portion of the cell culture medium.

While certain of the preferred embodiments of this invention have been described and specifically exemplified above, it is not intended that the invention be limited to such embodiments. Various modifications may be made without departing from the scope and spirit of the invention, as set forth in the following claims.

1. A single-use bag for containing and processing biological materials, said bag comprising: a body having a first end and an opposing second end, said ends bounding a compartment, and said body comprising at least one flexible sheet; an interior surface and an exterior surface, wherein the interior surface comprises a contact layer comprising low density polyethylene having a melt index from 0.8 to 2.5 g/10 min with a weight average molecular weight (Mw) greater than 200,000 Da and an Mz greater than 1,500,000 Da.

2. The single-use bag of claim 1, wherein the low density polyethylene does not comprise an antioxidant, a slip agent or an antiblock agent.

3. The single-use bag of claim 1, wherein the body comprises a two-dimensional pillow style bag or a three-dimensional bag.

4. The single-use bag of claim 1, wherein the body comprises at least three polymeric panels seamed together.

5. The single-use bag of claim 1, further comprising at least one fluid port mounted on the body so as to communicate with the compartment of the body.

6. The single-use bag of claim 1, wherein the compartment of the body has a volume of at least 500 milliliters.

7. The single-use bag of claim 1, wherein the flexible sheet comprises a laminated polymer film or an extruded polymer film, said laminated polymer film or extruded polymer film comprising two or more layers of different material.

8. The single-use bag of claim 7, wherein the extruded polymer film comprises at least three categorical layers including an internal contact layer that provides the inside surface of the single-use bag, an external layer that provides the outside surface of the single-use bag, and a gas barrier layer that is positioned between the internal contact layer and the external layer.

9. The single-use bag of claim 8, wherein the external layer comprises polyethylene, polypropylene, polyester, polyamide, or ionomer.

10. The single-use bag of claim 8, wherein the extruded polymer film comprises a thermoplastic elastomer external layer, a low density polyethylene contact layer, and an EVOH gas barrier layer disposed therebetween.

11. The single-use bag of claim 10, wherein thermoplastic elastomer comprises a styrenic block copolymer, polyolefin blend, elastomeric alloy, thermoplastic polyurethane, thermoplastic copolyetherester or thermoplastic copolyesteramide.

12. The single-use bag of claim 11, wherein thermoplastic elastomer comprises a thermoplastic co-polyether ester.

13. The single-use bag of claim 8, wherein the extruded polymer film comprises at least one additional layer including structure layer, bulking layer or adhesion layer positioned between the internal contact layer and the external layer.

14. The single-use bag of claim 8, wherein the gas barrier layer comprises EVOH between two layers of polyamide.

15. The single-use bag of claim 8, wherein the external layer or a structure layer comprises an ionomer.

16. The single-use bag of claim 8, wherein the flexible sheet comprises a multilayer film adhered to a substrate comprising a monoaxially or biaxially oriented film optionally coated with a barrier-enhancing agent.

17. The single-use bag of claim 1, for use in a biopharma process.

18. The single-use bag of claim 17, for use in bioreactors, cell culture, storage of media, sterile water for injection (WFI) or buffer solutions, or storage, final filling, mixing and final drug packaging of biological products.

19. The single-use bag of claim 1, for a single-use fluid container, reservoir, bag, bladder, pouch or membrane for storage, mixing, pumping, transfer or delivery of medicaments, pharmaceuticals, diluents, sera, intravenous solutions, whole blood, plasma, blood fractions or other fluids intended for treatment of humans or animals.

20. The single-use bag of claim 19, that is an intravenous (IV) bag, pump bladder, or ostomy pouch.
